(19) United States
(12) Patent Application Publication
(10) Pub. No.: US 2023/0028322 A1
(43) Pub. Date: Jan. 26, 2023

(54) COLD SOLUTION FOR INDUCING THERAPEUTIC HYPOTHERMIA

(71) Applicant: MIRAKI INNOVATION THINK TANK LLC, Cambridge, MA (US)

(72) Inventors: Christopher VELIS, Lexington, MA (US); Karen E. MILLER, South Dartmouth, MA (US); Emilia JAVORSKY, Watertown, MA (US); Rainuka GUPTA, Newton, MA (US); Tarik CHAUDHRY, Boston, MA (US)

(73) Assignee: MIRAKI INNOVATION THINK TANK LLC, Cambridge, MA (US)

(21) Appl. No.: 17/786,253

(22) PCT Filed: Dec. 28, 2020
(86) PCT No.: PCT/US20/67145

§ 371 Date: Jun. 16, 2022

Related U.S. Application Data
(60) Provisional application No. 62/955,699, filed on Dec. 31, 2019.

(51) Int. Cl. A61F 7/00; G16H 20/30

(52) U.S. Cl. CPC A61F 7/00; A61F 7/0085; G16H 20/30; A61F 2007/0063

(57) ABSTRACT

The present invention provides a cold solution and method for inducing therapeutic hypothermia. The cold solution can comprise liquid and/or solid ice particles.

### TECHNICAL FIELD

The invention relates to inducing therapeutic hypothermia using a cold solution.

### BACKGROUND

Therapeutic hypothermia, also called targeted temperature management, is the deliberate reduction of core body temperature, typically to a range of about 32 to 34 degrees Celsius. Inducing therapeutic hypothermia can be beneficial to cool tissue in the context of hyperthermia or to decrease the metabolic demand, for example, to preserve tissue and protect cells, neuroprotection and to mitigate ischemia.

Current systems and methods include cooling catheters and topical cooling methods such as cooling blankets and caps. Cooling catheters can cause infection and deep vein thrombosis. Topical cooling methods may require multiple and frequent exchanges of new cold applicators to be effective. Accordingly, improved systems and methods for inducing therapeutic hypothermia are needed.

### SUMMARY

The present invention provides a cold solution and method for inducing therapeutic hypothermia. A method of inducing therapeutic hypothermia in a subject can comprise delivering a cold solution to one or more treatment sites.

The delivering a cold solution to the one or more treatment sites can reduce the temperature of the one more treatment sites and/or reduce core body temperature of the subject. The delivering comprises delivering a cold solution to one or more treatment sites can be to treat one or more of the following: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia.

The delivering a cold solution to the one or more treatment sites decreases metabolic demand. The method can treat one or more of the following: cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, skin graft viability such as skin viability in a degloving injury, pulmonary embolism related ischemia, vascular surgery, acute trauma such as acute trauma to the musculoskeletal system, cryopreservation, preparing tissue or organisms for cryonics, suspended animation, tissue engineering such as to improve the viability of lab-grown tissues, and to augment effects of other neuroprotectants such as pharmacological or biological agents.

The delivering can comprise delivering a cold solution to tissue outside of a subject's body. The tissue can comprise a limb or organ.

The one or more treatment sites can comprise skin, blood, vessels such as the pericarotid artery or the jugular vein, soft tissue such as in the neck, peritoneum, GI tract, rectum, vagina, lung, heart, brain, nose, ear, mouth, a surgical site, antecubital fossa, vagus nerve, lumbar region, bladder, or a combination thereof.

The one or more treatment sites can comprise a heart. The one or more treatment sites can comprise a pericardial sac. The method can further comprise draining at least portion of a pericardial fluid. The method can further comprise placing a volume of cold solution in proximity to the heart. A volume of cold solution can be delivered transesophageally.

The one more treatment sites can comprise a brain. A volume of cold solution can be delivered via an intracranial opening. A volume of cold solution can be delivered to a location proximate a membrane and a bone.

The method can further comprise analyzing a subject, wherein the analysis is conducted by measuring the one or more treatment sites, an area surrounding the one or more treatment sites, or a combination thereof. The measuring can be conducted through visual measuring, measuring using imaging, computer assisted measuring, artificial intelligence assisted measuring, or a combination thereof.

The method can further comprise obtaining information from a subject prior to, during, and/or after administration of the cold solution, providing the information to a computer or artificial intelligence system, and utilizing the computer or artificial intelligence system to create a treatment plan the subject.

The method can further comprise removing at least a portion of the cold solution from the target tissue. The removing can comprise withdrawing the cold solution via a syringe. The removing can comprise withdrawing the cold solution via suction. The removing can comprise absorbing the cold solution.

The delivering can comprise injecting the cold solution to the one or more treatment sites. The injecting can be performed via a delivery device. The delivery device can comprise a cannula. The delivery device can comprise a syringe having a single needle. The syringe can comprise an array of needles. The array of needles can be expandable. The delivery device can comprise a syringe having one or more fenestrated needles. The delivery device can comprise a balloon. The balloon can be configured to protect non-target tissue from exposure to the cold solution. The balloon can be configured to contain the cold solution prior to delivery. The delivery device can also be configured to generate the cold solution. The injecting can comprise injecting one or more boluses of cold solution. The injecting can comprise injecting a cold solution in a pattern.

The delivering can comprise circulating a cold solution proximate to the one or more treatment sites. The cold solution can be circulated via an outerwear system.

The delivering can comprise topically contacting the cold solution to the one or more treatment sites.

The delivering can comprise inhaling the cold solution.

The delivering can comprise ingesting the cold solution.

The delivering can comprise inserting the cold solution to the one more treatments sites via an incision.

The delivering can comprise preconditioning the one or more treatment sites prior to a prolonged period of ischemia and/or postconditioning the one or more treatment sites after a prolonged period of ischemia. The preconditioning and/or postconditioning can comprise controlled cooling and rewarming of the one or more treatment sites. The controlled cooling and rewarming can comprise delivering the cold solution in a cyclical or pulsatile manner.

The method can further comprise administering at least one antioxidant.

The method can further comprise sensing a temperature of a subject. The method can further comprise delivering the cold solution based on the temperature of the subject exceeding a threshold.

The method can further comprise generating the cold solution. The method can further comprise generating the cold solution on demand.

The method can further comprise administering an anesthetic prior to and/or during the delivery of the cold solution.

A method can comprise delivering a cold solution to an agricultural product.

A method can comprise delivering a cold solution to lab grown meat.

A cold solution for inducing therapeutic hypothermia can comprise water and optionally one or more additives. The cold solution can comprise one or more of a salt, a sugar or a thickener. The cold solution can comprise one or more therapeutic agents. The cold solution can comprise one or more of an antioxidant, an aesthetic, a vasoconstrictor, an antibacterial, and a neuroprotectant.

The cold solution can comprise an osmolality of less than about 2,200 milli-Osmoles/kilogram. The cold solution can comprise an osmolality of less than about 1,000 milli-Osmoles/kilogram. The cold solution can comprise an osmolality of less than about 600 milli-Osmoles/kilogram. The cold solution can comprise an osmolality about 900 to about 2,200 milli-Osmoles/kilogram.

The cold solution can comprise a pH of about 4.5 to about 9.

The cold solution can comprise no ice. The cold solution can comprise ice particles at a concentration of about 2% to about 70%. The cold solution can comprise ice particles at a concentration of about 20% to about 50%. The cold solution can comprise ice particles at a concentration of about 71% to about 100%. The cold solution can comprise ice particles at a concentration of at least about 95%. The cold solution can comprise substantially ice.

The cold solution can comprise a temperature of about −25 degrees Celsius to about 10 degrees Celsius. The cold solution can comprise a temperature of about −6 degrees Celsius to about 0 degrees Celsius. The cold solution can comprise a temperature of at least 0 degrees Celsius. The cold solution can comprise a temperature of at least 10 degrees Celsius.

### BRIEF DESCRIPTION OF THE DRAWINGS

FIG. 1 is a view of exemplary treatment sites in a subject.

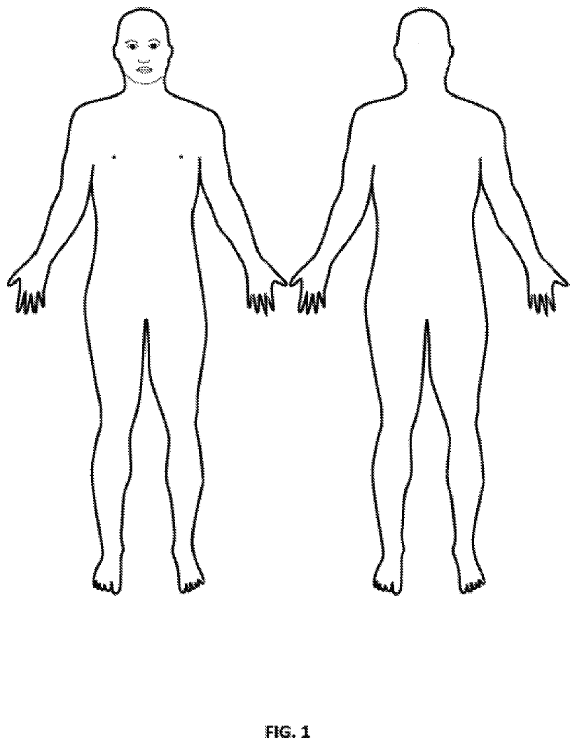

FIG. 2 is a view of an example device for delivering a cold solution.

FIG. 3 is a diagram a cold solution being delivered.

FIG. 4 is a view of an example device for delivering a cold solution.

FIG. 5 is a diagram of an approach to generating a cold solution inside a patient's body.

FIG. 6 is a view of a generating end of a point of delivery generation device for forming a cold solution from liquid water and solid water.

FIG. 7A is a view of a generating end of another point of delivery generation device for forming a cold solution from liquid water and solid water.

FIG. 7B is a sectional view of the generating end of FIG. 7A.

FIG. 8 is a view of a generating end of a point of delivery generation device for forming solid water from a first supply of liquid water and then forming a cold solution from the solid water and a second supply of liquid water.

FIG. 9 is a view of a generating end of a point of delivery generation device for forming a cold solution from supercooled water and ice pellets.

FIGS. 10A and 10B are views of a generating end of a point of delivery generation device for forming a cold solution inside a balloon.

FIG. 11 is a view of a point of delivery generation device for generating and replenishing cold solution.

FIG. 12 is a view of a point of delivery generation device having multiple working channels.

### DETAILED DESCRIPTION

The present invention provides a cold solution and method for inducing therapeutic hypothermia. It may be beneficial to induce therapeutic hypothermia to cool tissue in the context of hyperthermia (elevated body temperature) or to decrease metabolic demand, for example, to preserve tissue and protect cells, in neuroprotection, and to mitigate ischemia. The cold solution and method can be used to treat a subject such as a human or an animal subject.

Exemplary applications where it may be beneficial to cool tissue include: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia; and to alter the gut microbiome.

Exemplary applications where it may be beneficial to decrease metabolic demand include: cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, skin graft viability such as skin viability in a degloving injury, pulmonary embolism related ischemia, vascular surgery, acute trauma such as acute trauma to the musculoskeletal system, preservation, preparing tissue or organisms for cryonics, suspended animation, tissue engineering such as to improve the viability of lab-grown tissues, and to augment effects of other neuroprotectants such as pharmacological or biological agents.

A method of the present invention includes delivering a cold solution to one or more treatment sites to induce hypothermia. By providing a cold solution to the one or more treatment sites, the temperature of the treatment site and core body temperature is reduced. In some cases, inflammation is reduced at the treatment site. The cold solution can include a liquid and/or solid ice particles. For example, the cold solution can be substantially liquid, substantially solid, or a slurry, comprising both liquid and solid ice particles. The cold solution can be delivered by any suitable method based on the location of the treatment site and/or therapeutic application. For example, the cold solution can be injected, ingested, inhaled, inserted via an incision, or topically applied. An advantage of the present invention includes the ability to select the composition of the cold solution and delivery method based on the treatment and desired outcome.

The one or more treatment sites can include the skin 1, blood, vessels such as the carotid artery 2, the jugular vein 3, soft tissue such as in the neck 4, peritoneum 5, GI tract 6, rectum 7, vagina 8, lung 9, heart 10, brain 11, nose 12, ear 13, mouth 14, a surgical site 15, access to a vessel via the antecubital fossa 16, vagus nerve 17, lumbar region 18, and bladder 19.

Delivering a cold solution to the skin, for example, in topical applications can be useful in any of the above listed applications. A cold solution can be delivered topically via any suitable method, for example, via an applicator or container containing the cold solution, such as any wearable device as described below.

Delivering a cold solution to blood, a vessel, and/or soft tissue can be useful in any of the following applications: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia, to alter the gut microbiome, cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, skin graft viability such as skin viability in a degloving injury, pulmonary embolism related ischemia, vascular surgery, acute trauma such as acute trauma to the musculoskeletal system, cryopreservation, preparing tissue or organisms for cryonics, suspended animation, and to augment effects of other neuroprotectants such as pharmacological or biological agents. A cold solution can be delivered to blood, a vessel and/or soft tissue via any suitable method, for example, via injection. In some aspects, delivering a cold solution to blood can include injecting a cold solution to the blood via a vessel. In some aspects, delivering a cold solution to the blood can include partially or completely replacing a subject's blood with a cold solution via a vessel, for example, in a suspended animation application. In some aspects, the blood can be cooled outside of the body, for example where the cold solution is applied to the blood outside of the body, similar to a hemodialysis treatment.

Delivering a cold solution to the peritoneum and/or the GI tract can be useful in any of the following applications: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia, to alter the gut microbiome, cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, transplant surgery such as to preserve organs, reattachment surgery such as to preserve limbs, fingers, toes and the like, pulmonary embolism related ischemia, vascular surgery, acute trauma such as acute trauma to the musculoskeletal system, cryopreservation, preparing tissue or organisms for cryonics, suspended animation, and to augment effects of other neuroprotectants such as pharmacological or biological agents. A cold solution can be delivered to the peritoneum or GI tract via any suitable method, for example, via injection, ingestion or nasogastric tube.

Delivering a cold solution to the rectum, vagina and/or bladder can be useful in the following applications: hyperthermia such as heat stress, heat stroke, or when the core temperature exceeds 40 degrees Celsius due to prolonged exposure to elevated temperatures or exertional heat stroke or to decrease metabolic demands during a surgical procedure to improve outcomes. A cold solution can be delivered to the rectum, vagina and/or bladder via any suitable method, for example, via injection.

Delivering a cold solution to one or both lungs can be useful in the following applications: cardiac arrest, cardiac surgery such as cardiopulmonary bypass, transplant surgery such as to preserve organs, pulmonary embolism related ischemia, vascular surgery, acute trauma such as acute trauma to the musculoskeletal system, and trauma such as hypovolemic shock. A cold solution can be delivered to the lungs via any suitable method, for example, via inhalation or bronchoscope.

Delivering a cold solution to the heart can be useful in the following applications: cardiac arrest, cardiac surgery such as cardiopulmonary bypass, transplant surgery such as to preserve organs, pulmonary embolism related ischemia, vascular surgery, acute trauma such as acute trauma to the musculoskeletal system, and trauma such as hypovolemic shock. In some aspects, delivering a cold solution to the heart can include delivering the cold solution to the pericardial sac. In these aspects, the pericardial fluid surrounding the heart can be drained (partially or completely), and a cold solution can be delivered to the pericardial sac, e.g., injected to the pericardial sac via the intercostal space. For example, a volume of up to about 500 cc can be injected into the pericardial sac. In some aspects, the heart can be cooled by placing a volume of cold solution in proximity to the heart. For example, the heart can be cooled transesophageally by placing a device comprising a balloon (described below) comprising a cold solution in the esophagus.

Delivering a cold solution to the brain can be useful in the following applications: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia, to alter the gut microbiome, cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, pulmonary embolism related ischemia, vascular surgery, acute trauma such as acute trauma to the musculoskeletal system, cryopreservation, preparing tissue or organisms for cryonics, suspended animation, and to augment effects of other neuroprotectants such as pharmacological or biological agents. In some aspects, the brain can be cooled topically, for example, via a cap comprising circulating cold solution. In some aspects, a cold solution can be delivered to the brain via an intracranial opening, for example, via injection or topically during a brain surgery. In some aspects, a device comprising a balloon (described below) comprising a cold solution can be placed on the brain, for example, between the membrane and bone.

Delivering a cold solution to the nose and/or one or both ears can be useful in the following applications: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia, to alter the gut microbiome, cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, pulmonary embolism related ischemia, vascular surgery, acute trauma such as acute trauma to the musculoskeletal system, cryopreservation, preparing tissue or organisms for cryonics, suspended animation, and to augment effects of other neuroprotectants such as pharmacological or biological agents. In some aspects, a cold solution can be delivered to the nose and ears via any suitable method, for example, via injection.

Delivering a cold solution to the mouth can be useful to decrease metabolic demands during a surgical procedure to improve outcomes. In some aspects, a cold solution can be delivered to the mouth via any suitable method, for example, via injection or ingestion.

Delivering a cold solution to a surgical site can be useful to decrease metabolic demands during a surgical procedure to improve outcomes. In some aspects, a cold solution can be delivered to a surgical site via any suitable method, for example, via injection or a topical application.

Delivering a cold solution to the lumbar region can be useful in the following applications: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia, to alter the gut microbiome, cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, pulmonary embolism related ischemia, vascular surgery, acute trauma such as acute trauma to the musculoskeletal system, cryopreservation, preparing tissue or organisms for cryonics, suspended animation, and to augment effects of other neuroprotectants such as pharmacological or biological agents. In some aspects, a cold solution can be delivered to the lumbar region via any suitable method, for example, via injection.

In some aspects, the method includes delivering a cold solution to one or more treatment sites to minimize ischemia or reperfusion injury. In these aspects, inducing therapeutic hypothermia can reduce oxidative stress of reintroducing blood flow to an area after an ischemia by a pre-conditioning or a post-conditioning delivery of a cold solution. For example, a cold solution can be delivered to the affected tissue via controlled cooling and rewarming to pre-condition and/or post-condition the tissue prior to or after a prolonged period of ischemia. The cold solution can be delivered to the one or more treatment sites in a cyclical or pulsatile manner to minimize the effects of reperfusion injury. In these aspects, the cold solution can include an agent to mitigate oxidative damage such as an antioxidant. Exemplary antioxidants include α-tocopherol (Vitamin E), ascorbic acid (Vitamin C), β carotene (Vitamin A), selenium, ubiquinol, uric acid, lipoid acid, carotenoids, or enzymes such as catalases, glutathione peroxidase and superoxide dismutase (SOD).

In some aspects, the method includes reducing and/or maintaining a temperature of tissue outside of the body, for example in a limb or organ transplant application. In these aspects, a limb or organ can be placed in a container and attached to a cold solution generation system that provides continuous flow and agitation of a cold solution proximate to or in contact with the limb or organ. Such systems and methods are described in International Patent Application PCT/US2019/55633 filed on Oct. 10, 2019, which is incorporated by reference in its entirety herein. These systems allow for precise monitoring and maintenance of temperature.

In some aspects, the method includes reducing and/or maintaining a temperature of tissue inside of the body, for example, to preserve the organs of a deceased subject. In these aspects, the body can be flushed with a cold solution, for example via injection to the blood.

In some aspects, the method can be used in an application such as human hibernation, for example in space travel, where it may be useful to slow down the body's metabolism such that it reduces the body's use of resources and/or decreases the effects of radiation. Further, it is envisioned that a cold solution can be used in transhumanist applications, for example, for a subject that desires to freeze their body for preservation or other purposes.

In some aspects, the cold solution can be used in agricultural applications. For example, a cold solution can be used in laboratory applications such as to cool and/or maintain the temperature of lab grown meat. As another example, a cold solution can be used to maintain the temperature of agricultural products on transport.

In some aspects, the cold solution may include water. In some aspects, the cold solution may include water and one or more additives. In some aspects, the one or more additives are inactive, biocompatible ingredients, including any substance (at or below their respective concentrations) in the FDA GRAS list, which is incorporated by reference in its entirety herein. In some aspects, the additives comprise one or more of a salt, a sugar, and a thickener.

In some aspects, the cold solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the cold solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the cold solution comprises sucrose at about 5% by mass or lower, for example at about 5, 4.5, 4, 3.5, 3, 2.5, 2, 1.5, 1, 0.5, or 0% by mass. In some aspects, the cold solution comprises dextrose at about 5.6% by mass or lower, for example at about 5.5, 5, 4.5, 4, 3.5, 3, 2.5, 2, 1.5, 1, 0.5, or 0% by mass. In some aspects, the cold solution comprises mannitol at about 4.95% by mass or lower, for example at about 4.5, 4, 3.5, 3, 2.5, 2, 1.5, 1, 0.5, or 0% by mass. In some aspects, the cold solution comprises lactose at about 0.45% by mass or lower, for example at about 0.4, 0.35, 0.3, 0.25, 0.2, 0.15, 0.1, 0.05, or 0% by mass. In some aspects, the cold solution comprises sorbitol at about 4.7% by mass or lower, for example at about 4.5, 4, 3.5, 3, 2.5, 2, 1.5, 1, 0.5, or 0% by mass. In some aspects, the cold solution comprises glycerol at about 2% by mass or lower, for example at about 1.9, 1.8, 1,7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the cold solution comprises hetastarch at about 6% by mass or lower, for example at about 5.5, 5, 4.5, 4, 3.5, 3, 2.5, 2, 1.5, 1, 0.5, or 0% by mass. In some aspects, the cold solution comprises pectin at about 16.7% by mass or lower, for example at about 16, 15, 10, 9, 8, 7, 6, 5, 4, 3, 2, 1 or 0% by mass. In some aspects, the cold solution comprises polyethylene glycol at about 20% by mass or lower, for example at about 20, 15, 10, 9, 8, 7, 6, 5, 4, 3, 2, 1 or 0% by mass. In some aspects, the cold solution comprises gelatin at about 16% by mass or lower, for example at about 15, 10, 9, 8, 7, 6, 5, 4, 3, 2, 1 or 0% by mass. In some aspects, the cold solution comprises sodium methylcellulose at about 5% by mass or lower, for example at about 5, 4.5, 4, 3.5, 3, 2.5, 2, 1.5, 1, 0.5, or 0% by mass. In some aspects, the cold solution comprises a sodium alginate at about 5% by mass or lower, for example at about 5, 4.5, 4, 3.5, 3, 2.5, 2, 1.5, 1, 0.5, or 0% by mass. In some aspects, the cold solution comprises polyvinyl alcohol at about 5% by mass or lower, for example at about 5, 4.5, 4, 3.5, 3, 2.5, 2, 1.5, 1, 0.5, or 0% by mass. In some aspects, the cold solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the cold solution comprises Xanthan Gum at about 0.75% by mass or lower, for example at about 0.7, 0.6, 0.5, 0.4, 0.3, 0.2, 0.1, 0.05 or 0% by mass. In some aspects, the cold solution comprises CMC at about 0.75% by mass or lower, for example at about 0.7, 0.6, 0.5, 0.4, 0.3, 0.2, 0.1, 0.05 or 0% by mass. In some aspects, the cold solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises gum tracanth at about 1% by mass or lower, for example at about 0.9, 0.8, 0.7, 0.6, 0.5, 0.4, 0.3, 0.2, 0.1, 0.05 or 0% by mass. In some aspects, the cold solution comprises carbomer at about 1% by mass or lower, for example at about 0.9, 0.8, 0.7, 0.6, 0.5, 0.4, 0.3, 0.2, 0.1, 0.05 or 0% by mass.

Additional exemplary additives include bulking agents, such as sucrose, lactose, trehalose, mannitol, sorbitol, glucose, raffinose, glycine, histidine, PVP (K40); salts such as potassium, calcium, magnesium, hydrogen phosphate, hydrogen carbonate; buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetra acetic acid), disodium EDTA, calcium versetamide Na, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), cystein/cysteinateHCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol(thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate-anhydrous, sodium carbonate-anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, insitol, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-B-cyclodextrin, sulfobutylether-Bcyclodextrin); viscosity building agents such as celluloses such as sodium carboxymethylcellulose (CMC), hydroxyethylcellulose, hydroxypropylmethylcellulose, methylcellulose), acacia, gelatin, methyl cellulose, xanthan gum, polyethylene glycol, guar gum, locust bean gum, carrageenan, alginic acid, gelatin, carbopol, polyvinyl and pyrrolidone. Additives can be any of those found in Sougata Pramanick et al., “Excipient Selection in Parenteral Formulation Development,” 45(3) Pharma Times 65-77 (2013), which is incorporated herein by reference in its entirety.

In some aspects, the cold solution can include one or more therapeutic agents, for example, an antioxidant, an anesthetic, a vasoconstrictor, an antibacterial, and a neuroprotectant.

The cold solution can be delivered to a subject such as a human or an animal, therefore the solution can be sterile and have an osmolality and pH such that it does not harm target or non-target tissue. In some aspects, the cold solution may have an osmolality of less than about 2,200 milli-Osmoles/kilogram. In some aspects, the cold solution may have an osmolality of less than about 1,000 milli-Osmoles/kilogram. In some aspects, the osmolality may be less than about 600 milli-Osmoles/kilogram. In some aspects, the pH is between about 4.5 and about 9.

In some aspects, the cold solution is substantially liquid such as the cold solution described in PCT/US2019/55605 filed on Oct. 10, 2019, which is incorporated by reference in its entirety herein. The cold solution can be cooled or supercooled to a temperature just before spontaneous nucleation occurs. Alternatively, the cold solution can be cooled or supercooled to a temperature approximate to or lower than where spontaneous nucleation occurs, then warmed such that all ice particles melt prior to delivery to a subject. One example of a cold solution is water that is supercooled. Water normally freezes at 273.15 K (0° C. or 32° F.), but it can be supercooled at standard pressure down to its crystal homogeneous nucleation at almost 224.8 K (−48.3° C./−55° F.). The supercooling process requires that water be pure and free of nucleation sites. This can be done by processes like reverse osmosis or chemical demineralization. Rapidly cooling of water at a rate on the order of 10∧6 K/s avoids crystal nucleation and water becomes a glass, i.e., an amorphous (non-crystalline) solid. The temperature of the cold solution can be cooled to a temperature ranging from at about 10° C. to at about −50° C. One or more additives can be selected and included in the cold solution to change the freezing point of the cold solution.

In some aspects, the cold solution is substantially solid, i.e., substantially ice, such as the cold solution (and methods for making the cold solution) described in U.S. Provisional Patent Application Ser. No. 62/953,272 filed on Dec. 24, 2019, which is incorporated by reference in its entirety herein. For example, the cold solution can comprise ice at a concentration of about 71% to about 100%, including 71, 75, 80, 85, 90, 91, 92, 93, 94, 95, 96, 97, 98, 99, 99.5, 99.9 and 100%. In some aspects, the cold solution may comprise 95 to 100% of ice in a solid state, e.g., an ice composition. In some aspects, the cold solution comprising 95% to 100% ice in a solid state is an ice needle composition, which may be generated and/or delivered via a cannula such as a needle.

In some aspects, the cold solution is a slurry, including a liquid and solid ice particles, such as the slurry described in International Patent Applications PCT/US2019/54828 filed on Oct. 4, 2019 and PCT/US2015/047301 filed on Aug. 27, 2015, both of which are incorporated by reference in its entirety herein. Systems and methods for making a slurry are described in International Patent Application PCT/US2019/55633 filed on Oct. 10, 2019, which is incorporated by reference in its entirety herein. One or more additives can be selected to optimize flowability which is the ability of the slurry to flow through a device or within a subject. For example, flowability describes how easy it is for the slurry to move, either within the system for making the slurry, a delivery device for delivering the slurry such as a cannula, or within the body of a human subject. Flowability is dependent on several factors, including ice particle size, ice particle shape (as they relate to the configuration of the delivery device, for example, needle gauge) and viscosity.

The slurry includes ice particles, for example at a concentration of about 2% to about 70%. In some aspects, the ice concentration is about 20% to about 50%, for example, at about 30% to about 40%, for example, about 31, 32, 33, 34, 35, 36, 37, 38, or 39%. The ice particles can be substantially rounded and uniform in shape and size. Ice crystal size can be based upon the size of the delivery device, for example, an ice particle size of about 100 μm may allow injection through a needle having an inside diameter of about 1.0 mm or smaller. In some aspects, the ice particle size may be less than about 1 mm, or less than about 0.25 mm. In some aspects, the temperature of the slurry can range from about −25° C. to about 10° C., for example, from about −6° C. to about 0° C., for example, about −5° C., −4° C., −3° C., −2° C., −1° C.

In some applications, it may be beneficial to deliver a cold solution having a higher osmolality, for example in emergency applications, because the cold solution may maintain a colder temperature for a longer period of time. Example applications include organ cooling, cardiac arrest, and in the field or on transport to a healthcare facility. In these applications, the cold solution may have an osmolarity of about 900 milli-Osmoles/kilogram to about 2,200 milli-Osmoles/kilogram. The osmolality of the cold solution can be selected by selecting one or more additives and their respective concentrations, for example, a cold solution having a higher osmolality may include a higher concentration of the one or more additives.

In some applications, it may be beneficial to deliver a cold solution having a higher ice content, for example in an application where it is desired to cool a subject at a rapid rate or to a very cool temperature, such as below 20° C., for example in a suspended animation application.

In some applications, it may be beneficial to deliver a cold solution having a warmer temperature, for example when the treatment site comprises lipid rich cells such as the heart and brain to minimize the effects of the destruction of lipid rich cells via cooling. In these applications, the cold solution may be warmer than about 0° C., for example, warmer than about 10° C.

The type of cold solution(s) (i.e., substantially liquid, substantially solid, or slurry) and characteristics thereof (e.g., osmolality, volume, temperature, ice content, ice shape/size) can be selected based on the characteristics of the treatment site and desired outcome. A single treatment can include the delivery of one or more types of cold solution to one or more treatment sites via any suitable delivery method and any combination thereof.

Delivering a cold solution according to the present invention can be via any suitable means, for example, topically, via injection, ingestion, inhalation, incision, and any combination thereof. Any of the delivery methods described herein can be image guided.

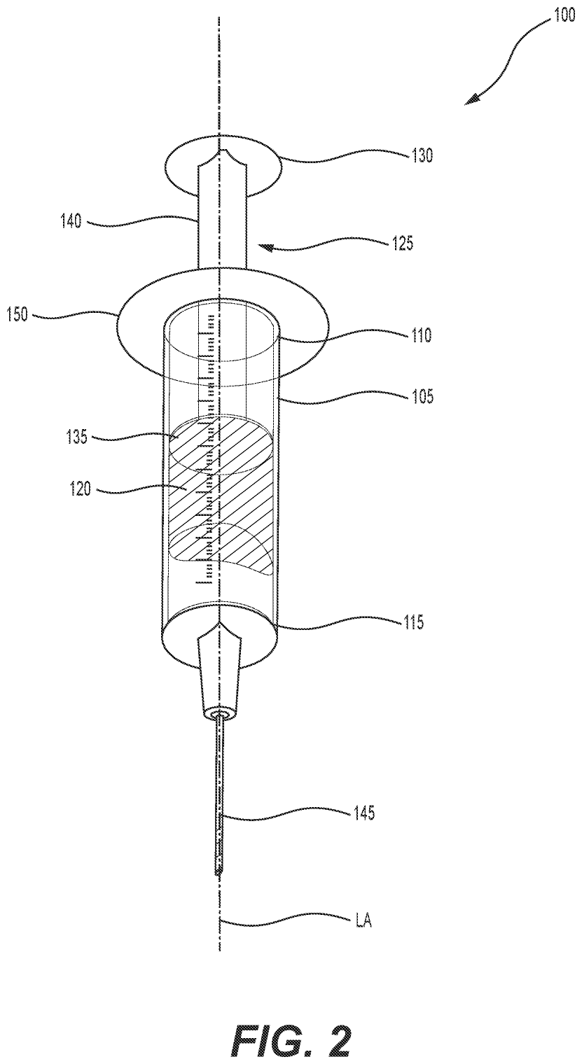

For example, delivering a cold solution can comprise injecting a cold solution via a delivery device. Any suitable delivery device may be used to deliver the cold solution to a subject. An exemplary device for delivering cold solution is generally shown in FIG. 2. The delivery device 100 includes a cylindrical member 105 having a first end 110 and a second end 115 along a longitudinal axis LA. The delivery device also includes an interior lumen 120 defined by the interior wall of the cylindrical member 105 and provided to receive and hold cold solution. The cylindrical member also includes a ledge 150, or flange, extending around the first end 110 out from the cylindrical member 105 along a plane that is orthogonal to the longitudinal axis LA. The ledge 150 also has an opening concentric with the interior lumen 120. The ledge helps facilitate handling and delivery of cold solution from the delivery device 100. In one embodiment, the delivery device 100 is a syringe-type device, for example, any suitable sterile syringe. The syringe can include a gauge size ranging from 8-25 G.

The cylindrical member 105 can be made of any type of biocompatible pharmacologically inert material suitable for use in holding and supplying fluids to be provided within a human body. Exemplary materials for the cylindrical member 105 include plastic, such as polyethylene or polypropylene, and glass. The delivery device 100 can be any size that suitable to hold one or more aliquots (doses) of cold solution for delivery to the desired tissue. As an example, the volume capacity of the delivery device 100 can be between 1 ml and 60 ml, although capacity outside of those volumes is also contemplated.

The delivery device 100 also includes a plunger 125 at least partially disposed within the interior lumen 120. The plunger 125 is configured to move in and out of the cylindrical member 105 through the first end 110. The plunger 125 includes a head 130, a plunging member 135, and a rod 140 extending between the head 130 and plunging member 135 along the longitudinal axis LA. The plunging member 135 is disposed along the rod 140 at a predetermined distance from the head 130. The delivery device 100 also includes at least one needle 145 extending from the second end 115. The needle 145 can comprise a gauge between 8 gauge and 25 gauge and a length between ¼ inch and 10 inches, such as about ¼ inch, ½ inch, 1 inch, 2 inches, 3 inches, 4 inches, 5 inches, 6 inches, 7 inches, 8 inches, 9 inches, or 10 inches. In some aspects, the cylindrical member 105 narrows or tapers to a small opening at the second end 115, where the small opening is configured to receive the needle 145. Preferably, the needle 145 is a hypodermic needle. Exemplary needle materials include, but are not limited to, stainless steel and carbon steel, with or without nickel plating.

The plunger 125, including the head 130 and the rod 140, can be any type of biocompatible, pharmacologically inert material suitable for coming in contact with fluids to be provided within a human body. Exemplary materials for the plunger 125 include plastic, such as polyethylene or polypropylene, and glass. With respect to the plunging member, a portion or all of the plunging member 135 can be a rubber material, such that a seal is formed between the sides of the plunging member 135 and the interior wall of the cylindrical member 105. The rubber material can be any rubber suitable for coming in contact with fluids to be provided to the human body, such as natural rubber latex or a synthetic rubber. In some aspects, the delivery device 100 can also include an agitator (not shown) disposed within the interior lumen 120 configured to mix the cold solution ingredients.

Once the cold solution is ready for delivery to tissue using the delivery device 100, the needle 145 is used to pierce the skin. Once the needle 145 is through the skin and positioned at or near the target tissue, the plunger 125 is forced downward toward the second end 115 of the cylindrical member 105. The force of the plunging member 135 on the cold solution forces the cold solution through the cylindrical member 105, out the needle 145, and into (or near) the treatment site. In one embodiment, more than one needle is provided at the second end 115 of the delivery device 100. The more than one needle can be provided in single row array, multiple row array, circular pattern, or any other conceivable arrangement.

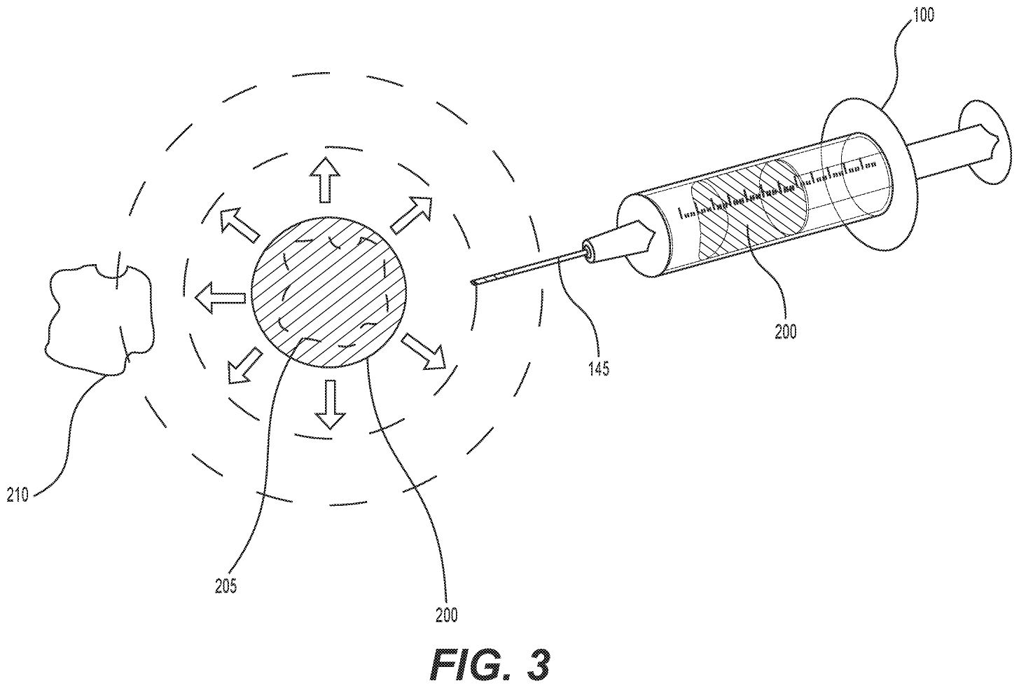

Referring to the procedure of FIG. 3, the device 100 of FIG. 2 is used to deliver cold solution 200 to a treatment site, tissue 205, for example, soft tissue located in a neck. In other examples, cold solution can be delivered using a syringe-type device, a catheter or a cannula. The needle 145 is inserted through the subject's skin and advanced to a location at or near the tissue 205 (shown in phantom line). The cold solution 200 is then delivered and cools the tissue 205 to achieve a therapeutic effect.

After delivery, an area affected by the cold solution 200 may expand to a size larger than the initial delivery site (shown in the figure as arrows radiating outwardly from the delivered cold solution 200 and dashed circles of increasing size). The cooling effect of the cold solution 200 is localized to the tissue 205 and possibly surrounding tissue, such as adjacent tissue 210. Accordingly, the treatment is targeted and precise. The cold solution 200 is sterile and biocompatible; and, as such, the cold solution 200 can be left in the body (e.g. no removal of the cold solution is necessary after cooling has been effected). In some aspects, the cold solution can be removed from the after the cooling has been effected. For example, after delivery to the heart and cooling has been effected, it may be desirable to remove at least a portion of the cold solution. The cold solution can be removed by any suitable means, for example via absorbing, e.g., via a sponge-like material, or withdrawing, e.g., using a syringe or suction.

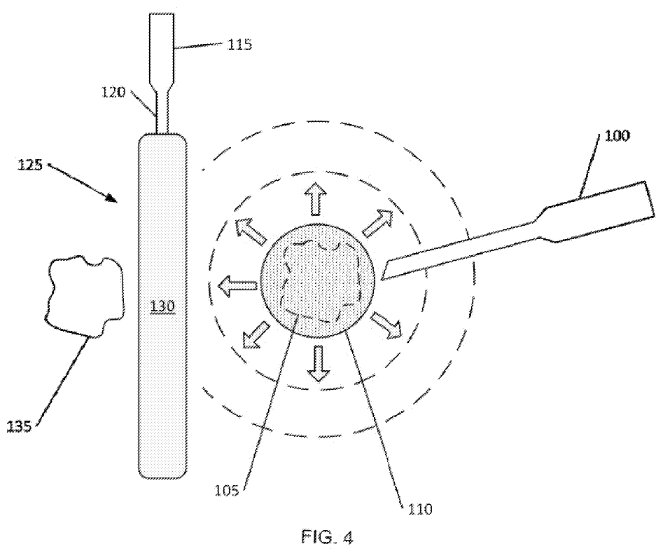

In some aspects, a cold solution containment device can be used in combination with the delivery device 100, for example, a device comprising a balloon configured for controlling the cooling effect of the cold solution, as shown in FIG. 4. As an example, a balloon deployment device 115 having an application cannula 120 is inserted through the patient's skin. At the distal end of the application cannula 120, there is a controlling end 125. The deployment device 115 is advanced until the controlling end 125 is at a location between the treatment site or target tissue 105 and an adjacent (surrounding) tissue 135. The controlling end 125 includes a balloon 130. While the balloon 130 is shown having a linear shape, it can have any shape, such as a ring that encircles the target tissue 105. In some aspects, the balloon 130 is filled with air to create a barrier between the adjacent tissue 135 and the spreading cold solution 110. The balloon 130 limits heat transferring from the adjacent tissue 135 to the cold solution 110. In some aspects, the delivery device 100 comprises a cannula such as a needle. In some aspects, the balloon deployment device is the delivery device, for example, balloon 130 can be filled with cold solution so as to deliver and contain the solution to a particular area.

In some aspects, delivering a cold solution to one or more treatment sites can include topically contacting the cold solution to the one or more sites. For example, the delivering of the cold solution can include contacting the treatment site and circulating the cold solution through a delivery device, for example, the delivery device described in FIG. 4 having a balloon. Alternatively, the cold solution can be circulated through a tubular member such as a catheter. This may be desirable for a treatment site that is more difficult to reach, for example, at a location in a lung. In another example, the delivering of the cold solution can include contacting the site and directly applying cold to the target tissue, for example, via a patch comprising the cold solution. The patch can include an adhesive configured to adhere to the treatment site. In some aspects, a cold solution can be delivered via an ice pack or a cooling blanket.

In some aspects, a cold solution can be delivered via an outerwear system worn by a subject, for example a human in hazardous situations such as a soldier in the field or a miner or firefighter. The outerwear system can be used to improve performance or provide a therapeutic benefit in an emergency situation such as overexposure to heat. The outerwear system can also be worn by a human at night to improve sleep by maintaining the core body temperature at or below a desired temperature. The outerwear can include outerwear including one or more of a shirt, pants, hat, socks and the like comprising tubing positioned on any portion or all of the outerwear. The system can be connected to an on-demand cold solution generator as described herein, for example, where the cold solution generator is stored in a backpack worn by the user. The cold solution can be circulated through the outerwear via a controller and can be circulated through a portion or the outerwear by including one or more valves to control the flow. In some aspects, the system can include one or more sensors in proximity to the subject, for example in a helmet, to initiate the generation and flow of cold solution within the system upon a detection of an increase in temperature or temperature exceeding a threshold. One or more sensors can be used in any of the disclosed embodiments to detect an increase in temperature or temperature exceeding a threshold.

In some aspects, delivering a cold solution to one or more treatment sites can include creating an incision and positioning the cold solution at or near the one or more sites. For example, when the cold solution is a substantially solid composition, an incision can be made proximate to the site and the substantially solid composition can be positioned at or near the site.

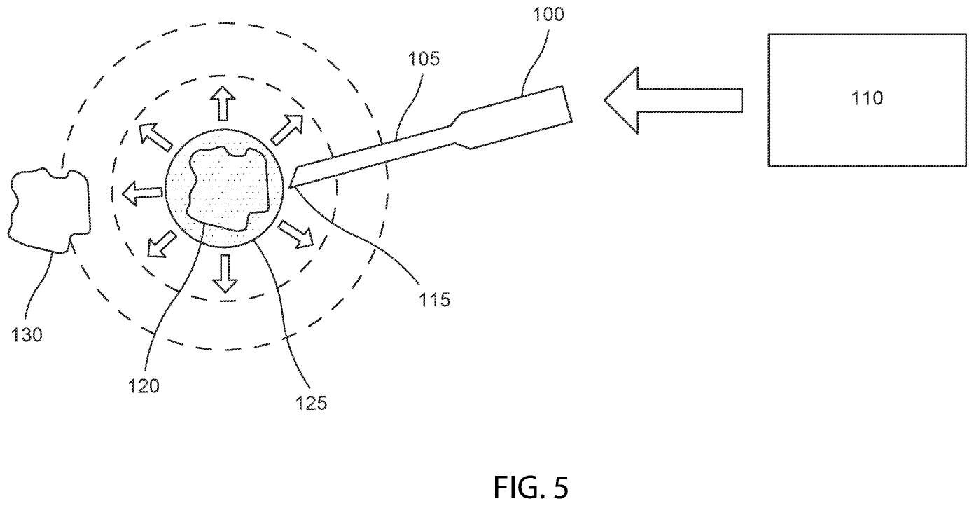

In some cases, for example, in an emergency situation, it may be advantageous to generate a cold solution on-demand in a short period of time. In some aspects, it may be desirable to generate at least 30 cc of cold solution in less than 10 minutes, for example, in five to eight minutes. In some aspects, it may be desirable to generate at least 10 cc of cold solution in less than one minute. Examples of such systems to generate a cold solution on demand or at the point of delivery are described in reference to FIGS. 5-12. These systems can be used to generate any type of cold solution described herein.

FIG. 5 shows an example of a point of delivery generation device 100 for making a cold solution inside a subject's body. The device 100 includes an application cannula 105 having a shape and size configured to be inserted through a subject's skin. The device 100 is fluidly coupled to a supply 110 providing components for making a cold solution. At the distal end of the application cannula 105, there is a generating end 115 for forming a cold solution from the components.

The point of delivery generation device 100 is used by inserting the application cannula 105 through the subject's skin and advancing the generating end 115 to a location at or near a target tissue or treatment site 120 (shown in phantom line). The target tissue 120 can, for example be subcutaneous adipose tissue. The solution ingredients, such as water and optionally one or more additives, are pumped or otherwise conveyed, separately, from the supply 110, through the application cannula 105, and out the generating end 115. At the generating end 115, the components interact with each other and form the cold solution 125 at or near the treatment site or target tissue 120.

The cooling effect of the cold solution 125 is localized to the target tissue 120 and possibly surrounding tissue, such as adjacent tissue 130. Accordingly, the treatment is targeted and precise. The cold solution is sterile and biocompatible; and, as such, the cold solution 125 can be advantageously left in the body (e.g. no removal of the cold solution is necessary after cooling has been effected).

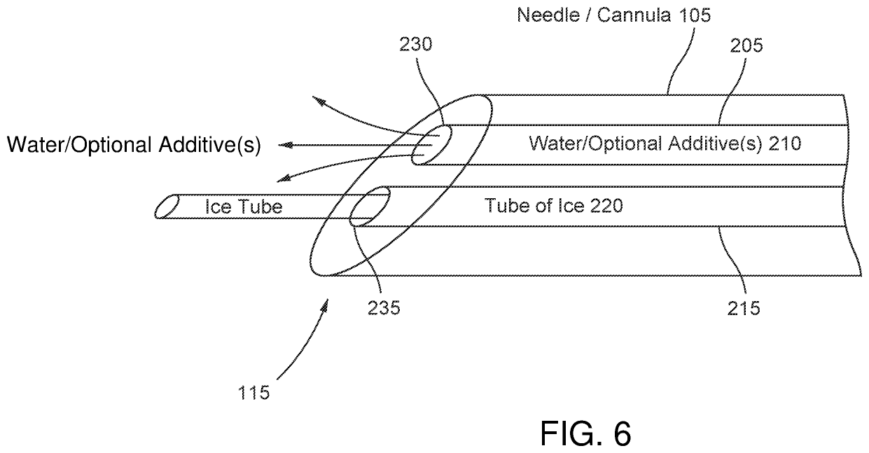

FIG. 6 shows an example of the generating end 115 for making a cold solution from mixing a solution comprising water and optionally one or more additives and water in a solid state, i.e., solid water. The cannula 105 houses a first delivery cannula 205 for supplying liquid water and optionally one or more additives 210 and a second delivery cannula 215 for supplying solid water (ice) 220. The distal end of the first delivery cannula 205 is open and forms a first outlet 230 for the liquid water and optionally one or more additives 210 to exit. The distal end of the second delivery cannula 215 is open and forms a second outlet 235 for the solid water 220 to exit. The outlets 230, 235 are arranged so that the liquid water and optionally one or more additives 210 and the solid water 220 mix together as they exit to form a cold solution. In some aspects, the second delivery cannula 215 can be configured to supply a cold solution including ice and one or more additives as described herein. In some aspects, second delivery cannula 215 can supply liquid water to generate a substantially liquid cold solution.

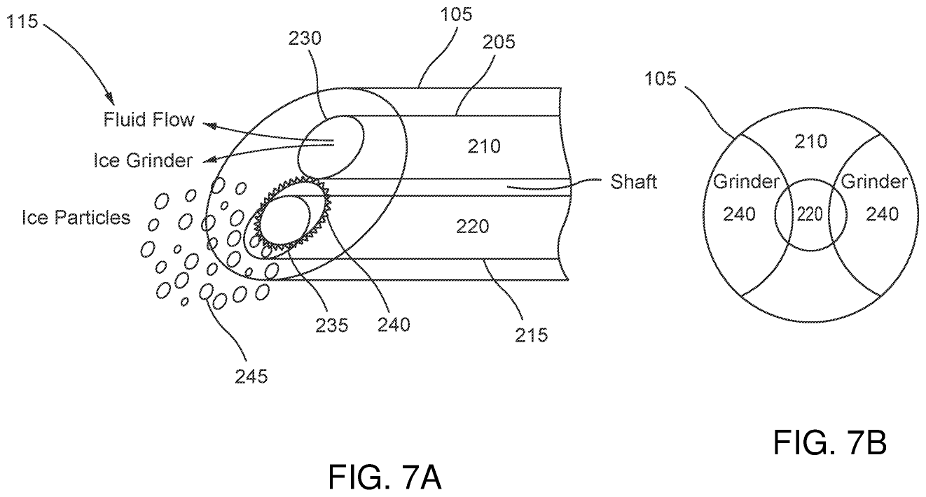

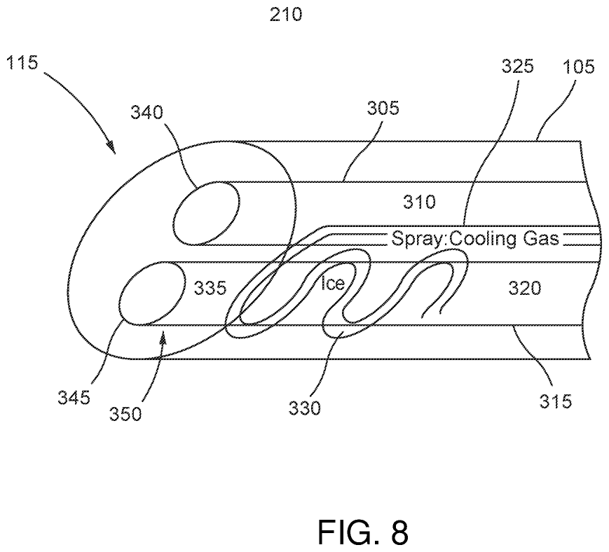

FIG. 7A shows an example of the generating end 115 for making a cold solution from mixing liquid water and optionally one or more additives, and solid water. This example is similar to the one described above with reference to FIG. 6 with the addition of a grinder 240 located in front of the second outlet 235. The arrangement of the grinder 240 with respect to the second outlet 235 is better seen in the cross-sectional view of FIG. 7B. As the solid water 220 emerges from the second delivery cannula 215, the grinder 240 breaks the solid water 220 into particles 245. The solution comprising liquid water and optionally one or more additives 210 exiting from the first delivery cannula 205 mixes with the particles 245 to form a cold solution. In another example (not shown), a vibrator can break the solid water into particles to combine with the solution comprising water and optionally one or more additives to form a cold solution at the point of delivery. In some aspects, second delivery cannula 215 can supply liquid water to generate a substantially liquid cold solution.

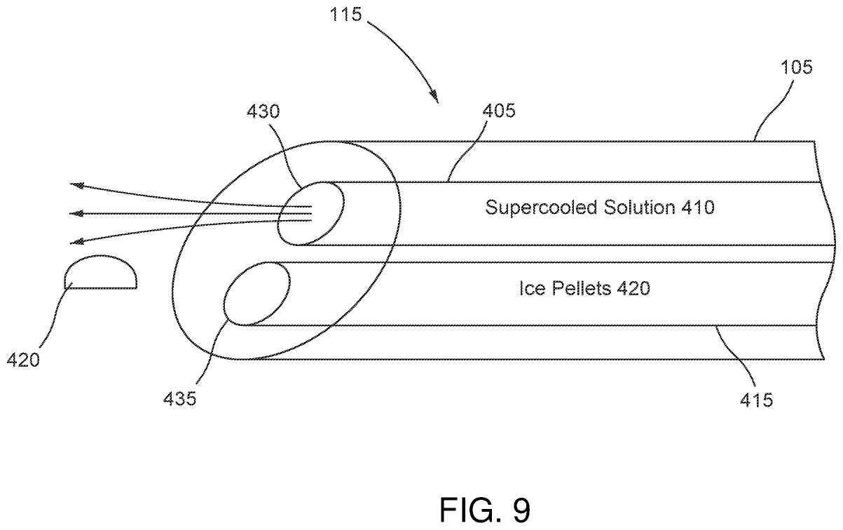

FIG. 8 shows another example of the generating end 115 for making a cold solution from mixing liquid water and optionally one or more additives, and solid water. The application cannula 105 houses a first delivery cannula 305 for providing a first supply of liquid water and optionally one or more additives 310, and a second delivery cannula 315 for providing a second supply of liquid water 320. As shown, the application cannula 105 further includes a gas line 325 for spraying a cooling gas 330 and freezing the second supply of water 320 into solid water 335.

The distal end of the first delivery cannula 305 is open forming a first outlet 340 for the first supply of liquid water and optionally one or more additives 310 to exit. The distal end of the second delivery cannula 315 is open forming a second outlet 345 for the solid water 335 to exit. In front of the second outlet 345, there is a grinder (or vibrator) 350 to break the solid water 335 into particles as it emerges from the second delivery cannula 315. The outlets 340, 345 are arranged so that the first supply of liquid water and optionally one or more additives 310 and the particles of solid water mix together to form a cold solution. In some aspects, gas line 325 is configured to spray a cooling gas 330 and cool the second supply of water 320 and reduce the temperature of water 320 without freezing water 320 to generate a substantially liquid cold solution.

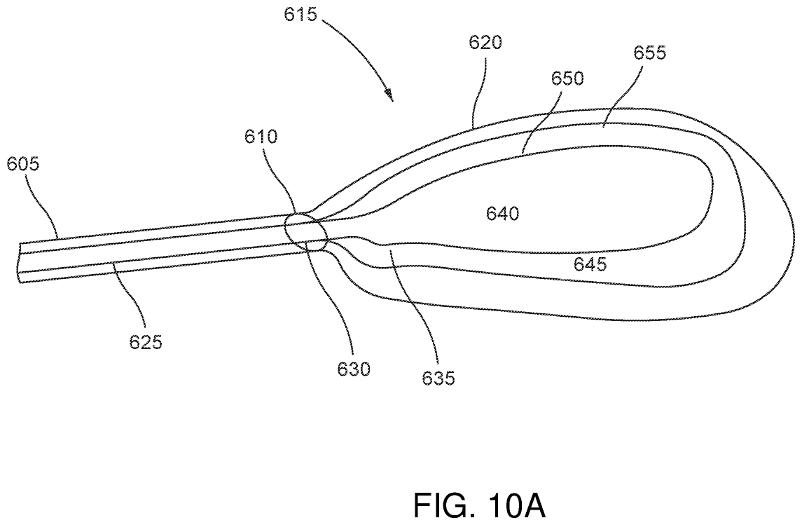

FIG. 9 shows an example of the generating end 115 for making a cold solution from crystalizing a supercooled solution comprising water and optionally one or more additives. The application cannula 105 houses a first delivery cannula 405 for supplying a supercooled solution comprising water and optionally one or more additives 410. Water normally freezes at 273.15 K (0° C. or 32° F.), but it can be “supercooled” at standard pressure down to its crystal homogeneous nucleation at almost 224.8 K (−48.3° C./−55° F.). The freezing point of the solution may vary depending upon the presence of one or more additives. The supercooling process requires that water be pure and free of nucleation sites. This can be done by processes like reverse osmosis or chemical demineralization. Rapidly cooling water at a rate on the order of 10⁶ K/s avoids crystal nucleation and water becomes a glass, i.e., an amorphous (non-crystalline) solid.

The application cannula 105 further houses a second delivery cannula 415 for supplying ice pellets 420, which serves as nucleation sites for the crystallization process. The distal end of the first delivery cannula 405 is open and forms a first outlet 430 for the supercooled water 410 to exit. The distal end of the second delivery cannula 415 is open and forms a second outlet 435 for the ice pellets 420 to exit. The outlets 430, 435 are arranged so that the supercooled water 410 interacts with the ice pellets 420 causing it to crystalize and form a cold solution. In some aspects, second delivery cannula 414 supplies liquid water to generate a substantially liquid cold solution.

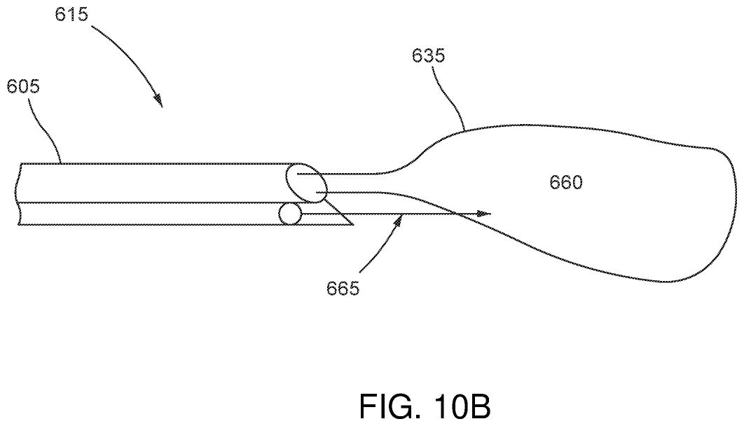

FIG. 10A shows another example of the point of delivery generation device. The device includes an application cannula 605 that is open at its distal end defining an outlet 610. A generating end 615 includes an outer balloon 620 disposed around the outlet 610. The application cannula 605 is in fluid communication with the interior volume of the outer balloon 620. The application cannula 605 includes a fluid delivery cannula 625. The application cannula 605 and the fluid delivery cannula 625 share a common longitudinal axis and can be said to be coaxially aligned.

The fluid delivery cannula 625 is open at its distal end defining a fluid outlet 630. The generating end 615 further includes an inner balloon 635 disposed around the fluid outlet 630. The fluid delivery cannula 625 is in fluid communication with an interior volume of the inner balloon 635, which is labeled 640 in the figure. The inner balloon 635 is located inside the outer balloon 620. As shown, the inner balloon 635 occupies a portion of the interior volume of the outer balloon 620 leaving a space or gap 645 between an outer wall of the inner balloon 635 (which is labeled 650 in the figure) and an inner wall of the outer balloon 620 (which is labeled 655 in the figure).

To generate a cold solution at the point of delivery, the application cannula 605 is inserted through a subject's skin and the generating end 615 is advanced to a location at or near a target tissue in much the same manner as described above with reference to FIG. 5. In this example, the outer balloon 620 and the inner balloon 635 are inserted into the patient's body in their uninflated state. The inner balloon 635 is then filled or inflated with a cool solution comprising cold water and optionally one or more additives that is supplied through the fluid delivery cannula 625.

Once the inner balloon 635 is filled with the cool solution, the outer balloon 620 is filled with a cooling gas or fluid, such as liquid nitrogen. The cooling gas fills the gap 645 between the inner balloon 635 and the outer balloon 620. This causes the cool solution in the inner balloon 635 to partially freeze and form a cold solution 660, as shown in FIG. 10B. (For clarity the outer balloon 620 is not shown in FIG. 10B.) The cold solution-filled inner balloon 635 can then be used to cool a target tissue. Alternatively, the inner balloon 635 can be ruptured by a retractable puncture needle 665 that extends beyond the application cannula 605 when extended as shown. Rupturing the inner balloon 635 releases the cold solution 660 at or near the target tissue. In some aspects, the cooling gas fills the gap between the inner balloon 635 and the outer balloon 620 to cool the solution in the inner balloon 635 to reduce the temperature of the solution without freezing the solution to generate a substantially liquid cold solution.

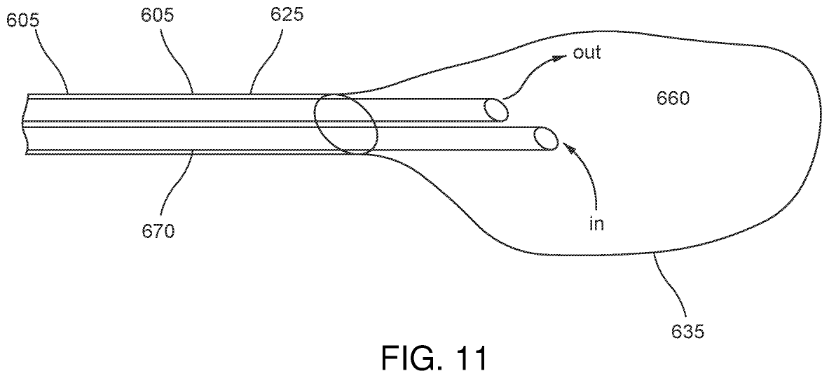

FIG. 11 shows another example of the point of delivery generation device for generating and replenishing a cold solution. This example is similar to the one described above with reference to FIG. 10 with the addition of a fluid return cannula 670. (For clarity the outer balloon 620 is not shown in FIG. 11.) The fluid return cannula 670 is housed within the application cannula 605 together with the fluid delivery cannula 625, as shown. The fluid return cannula 670 removes cold solution from the inner balloon 635 that is no longer at the desired temperature. Replenishing the “old” cold solution with “fresh” cold solution in this manner can accommodate for the eventually melting of cold solution. In some aspects, a clinician can administer a cold solution having a higher ice content, extending the period of cooling. This approach is particularly useful for a treatment requiring a long period of cooling.

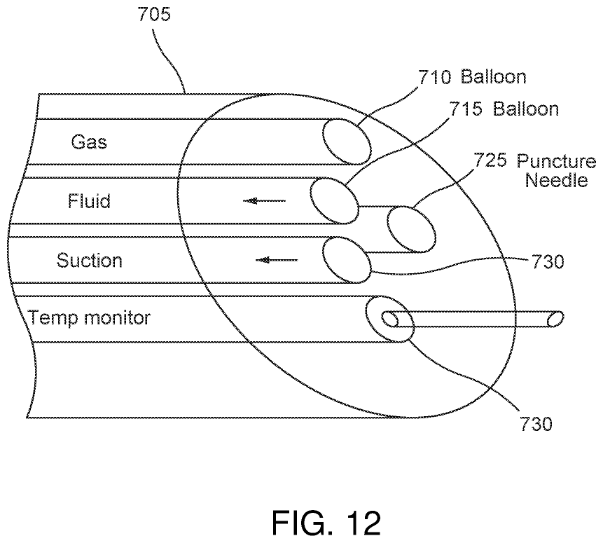

FIG. 12 shows an example point of delivery generation device having multiple cannulas or “working channels” to control the functions described above with reference to FIGS. 10A, 10B, and 11. The device includes an application cannula 705. The application cannula 705 houses a gas delivery cannula 710, a fluid delivery cannula 715, and a fluid return cannula 720, to continuously generate and replenish cold solution, as described above with reference to FIGS. 10A and 11. The application cannula 705 can also include a retractable puncture needle 725 to rupture a balloon filled with cold solution, as described above with reference to FIG. 10B. The application cannula 705 can further have a cold solution temperature monitor 730 for measuring the temperature of the cold solution.

FIGS. 5-12 describe on-demand or point of delivery cold solution generator devices that are combined with the delivery device. In other aspects, the on-demand cold solution generator device can be separate from the delivery device. In these aspects, the on-demand cold solution generator can include a port configured to dispense into a container or a delivery device such as a syringe. As described herein, the on-demand system can cool a solution via compressed gas, endothermic reaction or a standard refrigeration cycle. Additional systems and methods for on-demand cooling are described in US Publication No. 20180344074 which is incorporated by reference herein in its entirety.

An aspect of the invention includes a kit comprising one or more solutions comprising water and optionally one or more additives. When more than one solution is provided, the solutions may differ in their composition, including but not limited to the presence or absence of additives, the particular additives included and the amounts thereof, and the ice content. The kit may also include one or more of a mold for generating a substantially solid solution, a delivery device, a point-of-delivery generating device, a guide device, a balloon, a coil, a sterile bag or container (empty or pre-filled with a solution comprising water and optionally one or more additives), and a coating material, for example for coating a mold or a delivery device.

Any suitable volume of cold solution can be delivered based on the characteristics of the treatment site and desired effect. A treatment may include delivering a volume of cold solution to one or more treatment sites. For example, when a cold solution is delivered via injection, the site may be treated via one or more injection sites, i.e., puncture site, and one or more deposition sites. The deposition site is where the cold solution is deposited, regardless of the injection site, and may be a different site than the injection site or the same site. One or more treatments can be required to achieve a desired effect.

For example, where the treatment site is a larger area such as the abdomen and the delivery comprise injection, the amount of cold solution injected may be about 2L or less per injection site. In some examples, the amount of cold solution injected is about 1 mL to about 2L per injection site. For example, different patients have different amounts of subcutaneous fat, and therefore, some patients may require injection of greater amounts of cold solution in order to produce visible effects of reduction and removal of subcutaneous fat. Other patients may require multiple treatments to produce effects of removal or reduction of subcutaneous fat or tightening of the skin as a result of a collagen response.

As another example, where the treatment site is a smaller site such as soft tissue in the neck, the amount of cold solution injected may be about 1 mL to about 1L per injection site. For example, a cold solution can be delivered to the entire outer circumference of the neck or any portion or portions thereof.

In some aspects, the injection sites may form a pattern, such as a plow, fan, or grid-like pattern, or in a single bolus or multiple bolus injections. In some aspects, one injection site is used repeatedly, thereby reducing the number of injection sites and concomitant scarring potential. In a plow injection pattern, a single initial target injection site is used followed by a moving needle for additional deposition sites, for example in a linear pattern. In a fan injection pattern, deposition sites form an arc from 1 to 360 degrees. In a bolus injection, the cold solution is deposited in a single injection site.

The injection pattern and/or cold solution (including type and/or ingredients) can be determined based on the subject's profile, treatment plan (as described below), or based on the target site to be treated. For example, an injection pattern and/or volume may be selected to optimize consistency of temperature at the target site. In an embodiment, the injection pattern and/or volume is selected in order to achieve gradient cooling of the tissue proximate to or at a target site or injection site. Injection techniques, including the patterns described herein, are known to those of skill in the art.

Optionally, the method may include one or more of pre-, peri- and post-treatment monitoring such as imaging and/or performing one or more measurements. For example, the method can include determining one or more of the subject's core temperature, blood flow, stroke volume, heart rate, respiratory rate, central venous pressure, right ventricular pressure, pulmonary artery pressure, pulmonary capillary wedge pressure or left ventricular pressure. An indication of success would be an observation of a decrease in the core body temperature and an improvement in the associated condition.

Optionally, a treatment plan can be created for a subject, for example to determine one or more of type of cold solution, cold solution properties (for example, ingredients, tonicity and/or ice content), treatment sites(s), volume of cold solution to be delivered to each site, and delivery method(s) (e.g., ingestion, inhalation, injection, topical/contact, and/or incision). A single treatment can include delivering cold solution to one or more treatment or treatment sites. One or more cold solution types and corresponding volumes and delivery methods can be used for a single treatment site or multiple treatments sites in a single treatment. Factors considered in creating a treatment plan for a subject may comprise one or more of gender, height, body weight, body fat percentage, anatomy, lifestyle, vitals, medical history, lipid profiles, skin elasticity, medication, nutrition, supplements, demographic, fat saturation, sleep study observations, and the like. As an example, once a plan is created for the subject, the amount and type of cold solution to be administered can be adjusted based on one or more of the area or areas to be treated, the depth of injection, and the injection pattern to be used.

A computer or artificial intelligence system may be utilized to create a treatment plan for a subject by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy, lifestyle, the subject's vitals, medical history, lipid profiles, skin elasticity, medication, nutrition, supplements, demographic, fat saturation, imaging data, treatment data and fat loss data. Data may be measured by any suitable means. For example, data may be measured by any imaging method such as ultrasound, MRI, 3D photography, visual assessment, and the like.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing aspects are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of inducing therapeutic hypothermia in a subject comprising delivering a cold solution to one or more treatment sites.

2. The method of claim 1, wherein the delivering a cold solution to the one or more treatment sites reduces the temperature of the one more treatment sites and/or core body temperature of the subject.

3. The method of claim 2, wherein the delivering comprises delivering a cold solution to one or more treatment sites to treat one or more of the following: hyperthermia such as heat stress, heat stroke, or when the core temperature exceeds 40 degrees Celsius due to prolonged exposure to elevated temperatures or exertional heat stroke; overdose of or negative reaction to a pharmacologic agents such as anticholinergics, antihistamines, serotoninergics, psychomotor stimulants and diuretics; hormonal conditions such as thyrotoxicosis and pheochromocytoma; conditions of impaired sweating such as cholinergic neuropathy, autoimmune autonomic ganglionopathy, chronic idiopathic anhidrosis, botulism, generalized small fiber neuropathy, Sjogren syndrome, multiple system atrophy, Fabry's disease, bilateral cervical sympathectomy; and multiple sclerosis; disorders that increase thermogenesis such as status eplilepticus, neuroleptic malignant syndrome, and malignant hyperthermia.

4. The method of claim 1, wherein the delivering a cold solution to the one or more treatment sites decreases metabolic demand.

5. The method of claim 4, wherein the method treats one or more of the following: cardiac arrest, cardiac surgery such as cardiopulmonary bypass, trauma such as hypovolemic shock, acute ischemic stroke, traumatic brain injury, anoxic brain injury, neuroprotection, neonatal hypoxic-ischemic encephalopathy, spinal cord injury, anesthesia toxicity, preservation of residual hearing in cochlear implant surgery, transplant surgery such as to preserve organs, reattachment surgery such as to preserve limbs, fingers, toes and the like, skin graft viability such as skin viability in a degloving injury, pulmonary embolism related ischemia, vascular surgery, acute trauma such as acute trauma to the musculoskeletal system, cryopreservation, preparing tissue or organisms for cryonics, suspended animation, tissue engineering such as to improve the viability of lab-grown tissues, and to augment effects of other neuroprotectants such as pharmacological or biological agents.

6. The method of claim 1, wherein the delivering comprises delivering a cold solution to tissue outside of a subject's body.

7. The method of claim 6, wherein the tissue comprises a limb or organ.

8. The method of claim 1, wherein the one or more treatment sites comprise skin, blood, vessels such as the pericarotid artery or the jugular vein, soft tissue such as in the neck, peritoneum, GI tract, rectum, vagina, lung, heart, brain, nose, ear, mouth, a surgical site, antecubital fossa, vagus nerve, lumbar region, bladder, or a combination thereof.

9. The method of claim 1, wherein the one or more treatment sites comprise a heart.

10. The method of claim 9, wherein the one or more treatment sites comprise a pericardial sac.

11. The method of claim 10, further comprising draining at least portion of a pericardial fluid.

12. The method of claim 9, further comprising placing a volume of cold solution in proximity to the heart.

13. The method of claim 12, wherein a volume of cold solution is delivered transesophageally.

14. The method of claim 1, wherein the one more treatment sites comprise a brain.

15. The method of claim 14, wherein a volume of cold solution is delivered via an intracranial opening.

16. The method of claim 14, wherein a volume of cold solution is delivered to a location proximate a membrane and a bone.

17. The method of claim 1, further comprising analyzing a subject, wherein the analysis is conducted by measuring the one or more treatment sites, an area surrounding the one or more treatment sites, or a combination thereof.

18. The method of claim 17, wherein the measuring is conducted through visual measuring, measuring using imaging, computer assisted measuring, artificial intelligence assisted measuring, or a combination thereof.

19. The method of claim 1, further comprising obtaining information from a subject prior to, during, and/or after administration of the cold solution, providing the information to a computer or artificial intelligence system, and utilizing the computer or artificial intelligence system to create a treatment plan the subject.

20. The method of claim 1, further comprising removing at least a portion of the cold solution from the target tissue.

21. The method of claim 20, wherein the removing comprises withdrawing the cold solution via a syringe.

22. The method of claim 20, wherein the removing comprises withdrawing the cold solution via suction.

23. The method of claim 20, wherein the removing comprises absorbing the cold solution.

24. The method of claim 1, wherein the delivering comprises injecting the cold solution to the one or more treatment sites.

25. The method of claim 24, wherein the injecting is performed via a delivery device.

26. The method of claim 25, wherein the delivery device comprises a cannula.

27. The method of claim 26, wherein the delivery device comprises a syringe having a single needle.

28. The method of claim 27, wherein the syringe comprises an array of needles.

29. The method of claim 29, wherein the array of needles is expandable.

30. The method of claim 25, wherein the delivery device comprises a balloon.

31. The method of claim 30, wherein the balloon is configured to protect non-target tissue from exposure to the cold solution.

32. The method of claim 30, wherein the balloon is configured to contain the cold solution prior to delivery.

33. The method of claim 25, wherein the delivery device is also configured to generate the cold solution.

34. The method of claim 24, wherein the injecting comprises injecting one or more boluses of cold solution.

35. The method of claim 24, wherein the injecting comprises injecting a cold solution in a pattern.

36. The method of claim 1, wherein the delivering comprises circulating a cold solution proximate to the one or more treatment sites.

37. The method of claim 36, wherein the cold solution is circulated via an outerwear system.

38. The method of claim 1, wherein the delivering comprises topically contacting the cold solution to the one or more treatment sites.

39. The method of claim 1, wherein the delivering comprises inhaling the cold solution.

40. The method of claim 1, wherein the delivering comprises ingesting the cold solution.

41. The method of claim 1, wherein the delivering comprises inserting the cold solution to the one more treatments sites via an incision.

42. The method of claim 1, wherein the delivering comprises preconditioning the one or more treatment sites prior to a prolonged period of ischemia and/or postconditioning the one or more treatment sites after a prolonged period of ischemia.

43. The method of claim 42, wherein the preconditioning and/or postconditioning comprises controlled cooling and rewarming of the one or more treatment sites.

44. The method of claim 43, wherein the controlled cooling and rewarming comprises delivering the cold solution in a cyclical or pulsatile manner.

45. The method of claim 44, further comprising administering at least one antioxidant.

46. The method of claim 1, further comprising sensing a temperature of a subject.

47. The method of claim 48, further comprising delivering the cold solution based on the temperature of the subject exceeding a threshold.

48. The method of claim 1, further comprising generating the cold solution.

49. The method of claim 1, further comprising generating the cold solution on demand.

50. The method of claim 1, further comprising administering an anesthetic prior to and/or during the delivery of the cold solution.

51. A method comprising delivering a cold solution to an agricultural product.

52. A method comprising delivering a cold solution to lab grown meat.

53. A cold solution for inducing therapeutic hypothermia comprising water and optionally one or more additives.

54. The cold solution of claim 53, wherein the cold solution comprises one or more of a salt, a sugar or a thickener.

55. The cold solution of claim 55, wherein the cold solution comprises one or more therapeutic agents.

56. The cold solution of claim 53, wherein the cold solution comprises one or more of an antioxidant, an aesthetic, a vasoconstrictor, an antibacterial, and a neuroprotectant.

57. The cold solution of claim 53, wherein the cold solution comprises an osmolality of less than about 2,200 milli-Osmoles/kilogram.

58. The cold solution of claim 53, wherein the cold solution comprises an osmolality of less than about 1,000 milli-Osmoles/kilogram.

59. The cold solution of claim 53, wherein the cold solution comprises an osmolality of less than about 600 milli-Osmoles/kilogram.

60. The cold solution of claim 53, wherein the cold solution comprises an osmolality about 900 to about 2,200 milli-Osmoles/kilogram.

61. The cold solution of claim 53, wherein the cold solution comprises a pH of about 4.5 to about 9.

62. The cold solution of claim 53, wherein the cold solution comprises no ice.

63. The cold solution of claim 53, wherein the cold solution comprises ice particles at a concentration of about 2% to about 70%.

64. The cold solution of claim 53, wherein the cold solution comprises ice particles at a concentration of about 20% to about 50%.

65. The cold solution of claim 53, wherein the cold solution comprises ice particles at a concentration of about 71% to about 100%.

66. The cold solution of claim 53, wherein the cold solution comprises ice particles at a concentration of at least about 95%.

67. The cold solution of claim 53, wherein the cold solution comprises substantially ice.

68. The cold solution of claim 53, wherein the cold solution comprises a temperature of about −25 degrees Celsius to about 10 degrees Celsius.

69. The cold solution of claim 53, wherein the cold solution comprises a temperature of about −6 degrees Celsius to about 0 degrees Celsius.

70. The cold solution of claim 53, wherein the cold solution comprises a temperature of at least 0 degrees Celsius.

71. The cold solution of claim 53, wherein the cold solution comprises a temperature of at least 10 degrees Celsius.
